(19) United States
(12) Patent Application Publication
(10) Pub. No.: US 2025/0037792 A1
(43) Pub. Date: Jan. 30, 2025

(54) COMPUTATIONAL ASSESSMENT OF GENETIC VARIANT QUALITY

(71) Applicant: Inivata Ltd., Babraham (GB)

(72) Inventor: Paula Sofia de Sa Freire Pritchett, Saffron Walden (GB)

(73) Assignee: Inivata Ltd., Babraham (GB)

(21) Appl. No.: 18/782,935

(22) Filed: Jul. 24, 2024

Related U.S. Application Data
(60) Provisional application No. 63/515,361, filed on Jul. 25, 2023.

(51) Int. Cl. G16B 20/20; G16B 40/20; G16H 10/40

(52) U.S. Cl. CPC G16B 20/20; G16B 40/20; G16H 10/40

(57) ABSTRACT

Methods and apparatus for assessing quality of a genetic variant for inclusion as a biomarker in a circulating tumour DNA (ctDNA) assay. The method includes receiving genetic variants associated with a sample collected from a patient, the set of genetic variants including a first genetic variant, determining values for a plurality of characteristics associated with the first genetic variant, providing the values for the plurality of characteristics as input to a trained machine learning (ML) model, the trained ML model being trained to output a quality of a genetic variant, the quality of the genetic variant representing a likelihood that the genetic variant is both somatic and will sufficiently amplify using amplicon sequencing, and including the first genetic variant in a panel of genetic variants for use in a ctDNA assay for the patient based on the quality of the first genetic variant output from the trained ML model.

### CROSS REFERENCE TO RELATED APPLICATIONS

This application claims the benefit under 35 U.S.C. § 119 (e) to U.S. Provisional Patent Application No. 63/515,361, filed Jul. 25, 2023, and titled, “COMPUTATIONAL ASSESSMENT OF GENETIC VARIANT QUALITY,” the entire contents of which is incorporated by reference herein.

### FIELD OF THE INVENTION

This disclosure relates to techniques for computational assessment of genetic variant quality for use in a tumour-informed assay.

### BACKGROUND

After treatment for cancer, a small number of cancer cells may remain within a patient who appears to be in remission. These residual cells are called “minimal residual disease” (MRD) and may become a cause of relapse. Assays (e.g., circulating tumour DNA (ctDNA) assays) for detecting MRD can employ a variety of approaches, including sequencing a patient's tumour tissue to identify tumour-informed genetic variants, which may be indicative of MRD when detected in a patient's cell-free DNA (cfDNA).

### SUMMARY

In some embodiments, there is provided data analysis techniques for selecting a set of genetic variants for use in creating a tumour-informed assay (e.g., a ctDNA assay) designed to detect a panel of genetic variants in a sample (e.g., a blood sample). The sensitivity of the tumour-informed assay may be based, at least in part, on how many of the genetic variants included in the panel can be detected in the sample. Identifying genetic variants that are both somatic and likely to sufficiently amplify (e.g., using amplicon sequencing) in a tumour-informed assay is challenging using existing techniques. As a result, a substantial proportion (e.g., ⅓) of the genetic variants identified using existing techniques tend to be poor quality (e.g., they are not detected in a patient blood sample). Some embodiments of the present disclosure relate to a computational process for assessing the quality of a genetic variant prior to its inclusion in a panel of genetic variants used to create a tumour-informed assay. For instance, the quality of a genetic variant may be determined based on an output of a trained machine learning model, which receives as input values for a plurality of characteristics of the genetic variant.

In some embodiments, a method of assessing quality of a genetic variant for inclusion as a biomarker in a circulating tumour DNA (ctDNA) assay is provided. The method includes receiving a set of genetic variants associated with a sample collected from a patient, the set of genetic variants including a first genetic variant, determining values for a plurality of characteristics associated with the first genetic variant, providing the values for the plurality of characteristics as input to a trained machine learning (ML) model, the trained ML model being trained to output a quality of a genetic variant, the quality of the genetic variant representing a likelihood that the genetic variant is both somatic and will sufficiently amplify using amplicon sequencing, and including the first genetic variant in a panel of genetic variants for use in a ctDNA assay for the patient based on the quality of the first genetic variant output from the trained ML model.

In one aspect, the trained ML model is a classification model trained to classify the genetic variant as a good genetic variant or as a poor genetic variant, wherein classification as a good genetic variant represents a high likelihood that the genetic variant is both somatic and will sufficiently amplify using amplicon sequencing. In another aspect, including the first genetic variant in a panel of genetic variants for use in a ctDNA assay for the patient based on the quality of the first genetic variant output from the trained ML model comprises including the first genetic variant when the first genetic variant is classified as a good genetic variant. In another aspect, the trained ML model is a random forest model. In another aspect, the trained ML model includes a neural network. In an other aspect, the trained ML model is gradient boosting decision trees model. In another aspect, including the first genetic variant in a panel of genetic variants for use in a ctDNA assay for the patient based on the quality of the first genetic variant output from the trained ML model comprises substituting a second genetic variant for the first genetic variant in the panel of genetic variants when the first genetic variant is classified as a poor genetic variant. In another aspect, the method further includes determining values for the plurality of characteristics for the second genetic variant, providing, for the second genetic variant, corresponding values for the plurality of characteristics as input to the trained ML model to determine a quality of the second genetic variant, and substituting the second genetic variant for the first genetic variant in the panel of genetic variants only when the quality of second genetic variant is classified as a good genetic variant.

In another aspect, when the first genetic variant is classified as a poor genetic variant, the method further includes outputting an indication of one or more reasons why the first genetic variant was classified as a poor genetic variant. In another aspect, the method further includes determining values for the plurality of characteristics for each of the genetic variants in the set of genetic variants, providing, for each of the genetic variants in the set of genetic variants, corresponding values for the plurality of characteristics as input to the trained ML model to determine a quality of the genetic variant, and outputting an indication that the set of genetic variants is of poor quality when more than a threshold number of genetic variants in the set is determined to have a poor quality.

In another aspect, the method further includes determining a number of genetic variants in the panel of genetic variants, and removing the first genetic variant from the panel of genetic variants when the quality of the genetic variant is determined to have a poor quality and the number of genetic variants in the panel is greater than a threshold number. In another aspect, the sample is a cancer sample. In another aspect, the plurality of characteristics include one or more of read depth characteristics, copy number characteristics, variant quality characteristics, posterior somatic probability characteristics, or mutational signature characteristics. In another aspect, the method further includes filtering the set of genetic variants to produce a filtered set of genetic variants, wherein the first genetic variant is included in the filtered set of genetic variants. In another aspect, the ctDNA assay is an amplicon sequencing assay. In another aspect, the method further includes testing a plasma sample from the patient using the ctDNA assay, and outputting an assay result. In another aspect, the plasma sample is a sample taken 1-12 months after surgery to remove cancerous tissue from the patient.

In some embodiments, a method of training a machine learning (ML) classification model to predict the quality of a genetic variant for inclusion as a biomarker in a circulating tumour DNA (ctDNA) assay is provided. The method includes receiving data for a plurality of genetic variants associated with cancer samples, wherein the data includes for each genetic variant of the plurality of genetic variants, values for characteristics associated with the genetic variant and a classification of whether the genetic variant is a good genetic variant or a poor genetic variant, wherein classification as a good genetic variant represents a high likelihood that the genetic variant is both somatic and will sufficiently amplify using amplicon sequencing, training, using the received data, the ML classification model to predict whether a new genetic variant not included in the plurality of genetic variants is a good genetic variant or a poor genetic variant, and outputting the trained ML classification model for use in classifying genetic variants in a sample.

In one aspect, the trained ML classification model is a random forest model. In another aspect, the trained ML classification model includes a neural network. In another aspect, the trained ML classification model is gradient boosting decision trees model. In another aspect, the sample is a cancer sample. In another aspect, the characteristics associated with the genetic variant include one or more of read depth characteristics, copy number characteristics, variant quality characteristics, posterior somatic probability characteristics, or mutational signature characteristics. In another aspect, the ctDNA assay is an amplicon sequencing assay.

In some embodiments, at least one hardware computer processor is provided. The at least one hardware computer processor may be programmed to perform any of the methods described herein.

In some embodiments, a computer readable medium is provided. The computer readable medium may be encoded with a plurality of instructions, that, when executed by at least one hardware computer processor perform any of the methods described herein.

In some embodiments, a tumour-informed assay for monitoring the presence of cancer in a patient is provided. The tumour-informed assay may be configured to detect at least one genetic variant from the output set of genetic variants according to any of the methods described herein.

### BRIEF DESCRIPTION OF DRAWINGS

Various non-limiting embodiments of the technology will be described with reference to the following figures. It should be appreciated that the figures are not necessarily drawn to scale.

FIG. 1 is a flowchart of a process for selecting a panel of genetic variants for use in a tumour-informed assay in accordance with some embodiments of the present disclosure.

FIG. 2 is a flowchart of a process for characterizing genetic variants in accordance with some embodiments of the present disclosure.

FIG. 3A is a schematic illustration of a quality control (QC) process that may be used to assess a quality of a genetic variant in accordance with some embodiments of the present disclosure.

FIG. 3B is a flowchart of a process for training a machine learning (ML) model using values for a plurality of characteristics and classifications of genetic variants in accordance with some embodiments of the present disclosure.

FIG. 4 is a flowchart of a process for training an ML model in accordance with some embodiments of the present disclosure.

FIG. 5 is a flowchart of a process for using a trained ML model to determine a quality of a genetic variant in accordance with some embodiments of the present disclosure.

FIGS. 6-7 show several charts depicting the differences in various characteristics between good quality genetic variants and poor quality genetic variants in accordance with some embodiments of the present disclosure.

FIG. 8A shows a chart depicting the number of variants differing between the results from a QC process and from model predictions in accordance with some embodiments of the present disclosure.

FIG. 8B shows a chart depicting the number of additional good quality genetic variants identified by model predictions to rescue certain panels in accordance with some embodiments of the present disclosure.

FIG. 9 is a barplot showing the number of variants predicted to be good quality variants and poor quality variants by a predictive model for several tumour samples in accordance with some embodiments of the present disclosure.

### DETAILED DESCRIPTION

Aspects of the technology described herein relate to techniques for assessing the quality of genetic variants prior to including them in a panel used to create a tumour-specific assay (e.g., a ctDNA assay for detecting MRD) for a patient. In a tumour-specific assay, a panel of a particular number (e.g., 12, 24, 48, 64, 100, 500, 1000, 5000) of genetic variants may be selected for use in the assay. Due in part to the heterogeneity of detectable genetic variants across cancer types, patients, and samples, it is often challenging to devise an assay that includes an optimal panel of genetic variants for a particular patient (e.g., a panel of genetic variants each of which will be detected in a sample from the patient if MRD is present). Some existing techniques for selecting genetic variants for inclusion in a panel may compare values for one or more characteristics of the genetic variant to predetermined threshold values and include or reject genetic variants from the panel based on whether the values for the one or more characteristics are above or below the predetermined threshold values. The inventors have recognized and appreciated that existing techniques for selecting genetic variants for inclusion in a panel may be improved by assessing the quality of a candidate genetic variant using a computational model (e.g., a machine learning model) trained to output a quality for the genetic variant based on values for a plurality of characteristics of the genetic variant. The quality of the genetic variant output from the trained machine learning model may then be used to inform a decision of whether to replace the genetic variant on the panel with an alternative genetic variant not initially included on the panel (e.g., because it was associated with a lower prioritization).

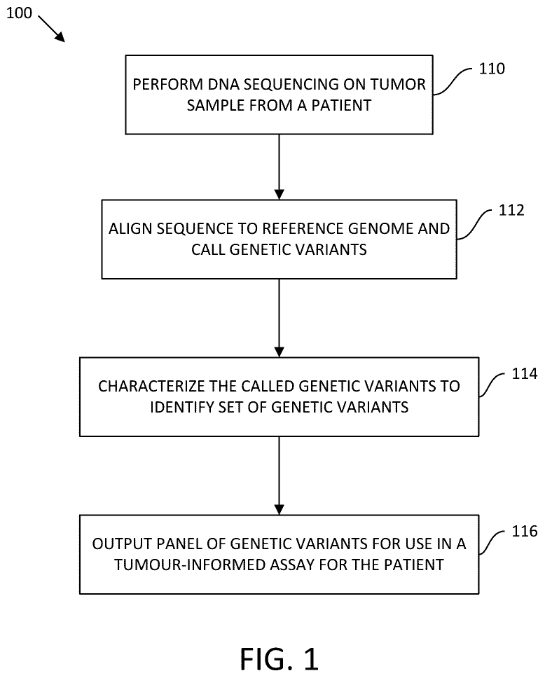

FIG. 1 is a flowchart of a process 100 for determining a panel of genetic variants for use in a tumour-informed assay in accordance with some embodiments of the present disclosure. Process 100 begins in act 110, where DNA sequencing is performed on a tumour sample from a patient. For instance, a tumour sample from a patient with cancer may be obtained during a resection of the tumour or tissue biopsy or obtained in some other manner. The obtained tumour sample may be prepared for DNA sequencing and may be sequenced (e.g., using whole genome sequencing or whole exome sequencing). Process 100 then proceeds to act 112, where the resulting sequence reads may be aligned to a human reference genome, and genetic variations (also referred to herein as “genetic variants”) may be called (e.g., using conventional DNA alignment software tools).

Process 100 then proceeds to act 114, where the called genetic variants may be characterized to identify a set of genetic variants that have particular values (e.g., within specified ranges) for characteristics of interest. In some embodiments, the characteristics of interest may include, but are not limited to, one or more of read characteristics (e.g., read depth), copy number characteristics, variant quality characteristics, posterior somatic probability characteristics, or mutational signature characteristics. Somatic genetic variants have been shown to be the most common cause of many cancers. In some embodiments, the called genetic variants are analyzed to determine which genetic variants are likely somatic variations (e.g., rather than germline variations) in the tumour, e.g. by comparing the tumour sample with a matched normal sample (such as white blood cells from the same patient). For instance, software tools such as Mutect2 (see https://gatk.broadinstitute.org/hc/en-us/articles/360037593851-Mutect2) may be used to identify genetic variants that are likely somatic variations. Mutect2 is a somatic variant caller that uses local assembly and realignment to detect single nucleotide variants (SNVs) and indels. Although software tools such as Mutect2 output values for a large number of characteristics, the inventors have recognized that not all characteristics of a genetic variant may be relevant to a determination of genetic variant quality. Accordingly, in some embodiments, values for a subset of the characteristics may be used. Examples of characteristics of interest output from software tools such as Mutect2 include, but are not limited to, GERMQ (Phred-scaled quality score that alternative alleles are not germline variants), DP_T (total read depth after filtering poor quality reads (e.g., reads with a missing MAPQ score or with bad mates are filtered)), REF_T (allele depth of the reference allele), ALT_T (allele depth of the alternative allele), and AF_T (allele fractions of the alternative allele).

As another example, characteristics of interest for called genetic variants may include, but are not limited to, tumour purity, copy number, loss of heterozygosity (LOH), classification of SNVs by somatic status, and clonality. For instance, software tools such as PureCN (see https://bioconductor.org/packages/release/bioc/html/PureCN.html) may be used to analyze the called genetic variants to identify values for one or more characteristics of the genetic variants. PureCN is a software tool for identifying somatic genetic variants using a Bayesian approach, and includes a list of common single nucleotide polymorphisms (SNPs) to label false positive genetic variants. Examples of characteristics output from software tools such as PureCN include, but are not limited to, Posterior Somatic Probability (posterior probability that a variant is a somatic mutation), Copy Number (maximum likelihood integer of copy number), EPR (label of somatic prioritization based on external annotations), T_FILTER (flag indicating which of a given set of filters the variant has failed or passed), Is_snp (indicates whether a variant coincides with a known SNP), and PureCN.FLAGGED (flag indicating variant has poor quality).

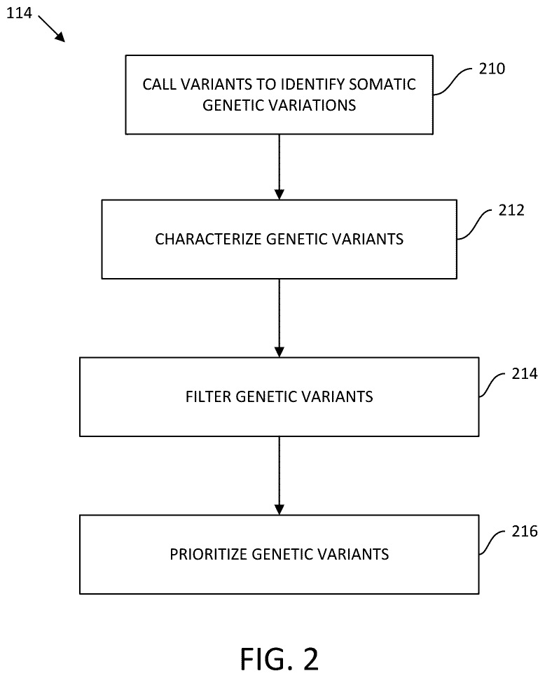

FIG. 2 is flowchart of a process that includes further details on processing in act 114 of process 100 in accordance with some embodiments. As shown in FIG. 2, act 114 may begin in act 210, where genetic variants are called (e.g., using Mutect2, PureCN and/or some other software tool) to identify likely somatic genetic variations in a sequenced sample. The process then continues to act 212, where the identified genetic variants are characterized by determining values for one or more characteristics of each of the genetic variants. For instance, software tools (e.g., Mutect2, PureCN) may output values for one or more characteristics of interest (examples of which are described herein) for each genetic variant that is called. In some embodiments, characterizing the genetic variants includes attempting to match the genetic variant with one or more mutational signatures for known somatic variants. In such embodiments, variants that match a mutational signature with high probability may be considered likely to be somatic genetic variants, whereas variants that do not match to a mutational signature may be considered less likely to be a somatic genetic variant. It should be appreciated that in some embodiments, the identification of genetic variants in act 210 and the characterization of genetic variants in act 212 may be performed together rather than as sequential act. The process shown in FIG. 2 may then proceed to act 214, where one or more of the genetic variants may be filtered based on particular criteria that are used to identify “good-quality” variants. For example, values for one or more characteristics determined in act 212 may be compared to one or more thresholds to separate “poor quality” from “good quality” variants. In some embodiments, filtering genetic variants in act 214 may not be performed and/or may be replaced by use of a trained machine learning model, as described in more detail below. Examples of matching variants to mutational signatures and calculating a probability that a variant was generated by exposure to a mutational signature can be found in U.S. Provisional Patent Application No. 63/439,769, the contents of which are hereby incorporated by reference in its entirety.

The process shown in FIG. 2 may then proceed to act 216, where the remaining genetic variants (e.g., after filtering or after being processed by a trained machine learning model) may be prioritized. In some embodiments, genetic variants may be prioritized based on one or more of region mappability, error rate predicted during calibration, or genomic diversity. Region mappability is a characteristic that describes whether the genetic variant occurs in repetitive regions in the genome and/or regions with low complexity. Genomic diversity is a characteristic that describes whether a genetic variant is observed on a sufficient number of different chromosomes, to mitigate effects of cancer evolution and/or sudden variant dropout. Any suitable technique for prioritizing genetic variants may be used. For example, a weighted average of clonality, region mappability and predicted error rate may be used to assign a prioritization to a genetic variant. As another example, genetic variants may be prioritized based on their location in the genome. For example, variants that are less accessible to short sequencing reads may be associated with a lower prioritization. As another example, genetic variants with lower predicted error rates may be associated with a higher prioritization. As described herein, the prioritization associated with a genetic variant may be used to select an initial set of variants for use in a panel and/or may be used, at least in part, to select alternative genetic variants (e.g., not included in the initial set), as needed.

Based on the identified values for one or more characteristics of the genetic variants and/or the associated prioritization values, a subset of genetic variants having particular values may be identified in act 114 (e.g., after characterization, filtering, and/or prioritization). For instance, one or more of the characteristics may be associated with threshold values, and called genetic variants having values for characteristics that are less than (or more than) the corresponding threshold values may be included in the subset of genetic variants. In some embodiments, the subset of genetic variants may be limited by a particular number (e.g., 50, 60, 70, 80, 90, 100) of variants. In some embodiments, an initial identified set of genetic variants may be further filtered or refined based on one or more prioritization criteria to produce a final set of genetic variants.

Process 100 then proceeds to act 116, where a panel of genetic variants for use in a tumour-informed assay may be output. For example, primers may be designed that target the flanking 5′ and 3′ regions of each genetic variant in the set of genetic variants output from act 114. The primers may be used in subsequent multiplex PCR reactions, such that the genetic variants are amplified when the tumour-informed assay is used to process further samples (e.g. blood samples) for a patient.

In some embodiments, the tumour-informed assay may be a liquid biopsy assay designed to analyze blood (e.g., plasma) samples to detect whether a patient has MRD. To reduce the number of blood samples that are required to test for MRD, it may be important to ensure that the genetic variants included in the panel for the tumour-informed assay are good quality genetic variants (e.g., they will be detectable in patient blood samples when the patient has MRD). If too many of the genetic variants included in the panel are not good quality variants, it may be decided, for example, to redesign the panel by including different genetic variants that have better quality. To evaluate the quality of genetic variants that have been selected for inclusion into a panel, a quality control (QC) process may be implemented to check for the presence of the variant in a tumour sample, but not in a non-tumour control sample.

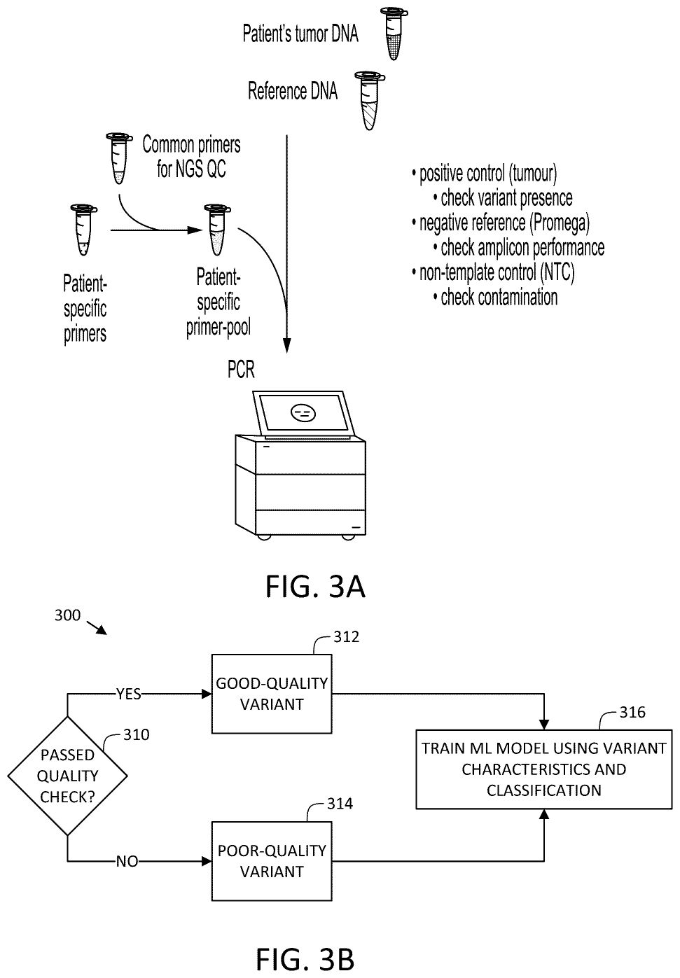

FIG. 3A schematically illustrates a QC process that may be used to evaluate the quality of genetic variants in accordance with some embodiments. As described above, after identifying a set of variants to include in a panel, primers may be designed to target the 5′ and 3′ regions of each genetic variant and the primers may be used to amplify those regions in the sample (e.g., using amplicon sequencing). In the QC process shown in FIG. 3A, DNA from a tumour sample (e.g., FFPE tumour tissue) may be tested against the patient-specific primer panel to determine whether variants in the panel are identified in the sample. If a genetic variant is not detected in the tumour DNA, the variant may be considered to be a poor-quality genetic variant and may be excluded from the assay. As shown in FIG. 3A, the input to the QC process may be an aliquot of DNA from the tumour sample, an aliquot of reference DNA lacking the variant of interest, and a non-template control to test whether each of the samples will amplify the variant of interest. When the region corresponding to the genetic variant of interest is amplified in the tumour sample, but not the reference sample or the control sample, the variant may be classified as a good genetic variant and may be included in the tumour-specific assay. When the region corresponding to the genetic variant of interest is not amplified in the tumour sample, or when it is amplified in both the tumour sample and the reference or non-template control samples, the variant may be classified as a poor-quality genetic variant, and it may not be included in the tumour-specific assay.

The inventors have recognized and appreciated that information obtained from a QC process such as the QC process shown in FIG. 3A may be used to train a computational model (e.g., a machine learning (ML) model) to be able to predict whether genetic variants having particular values for one or more characteristics (e.g., characteristics determined in act 114 of process 100) are likely to be classified as a good genetic variant (e.g., will pass the QC process) or a poor genetic variant (e.g., will fail the QC process). Additionally, because the QC process also attempts to amplify the genetic variants in the panel, a measure of how well each genetic variant amplifies (e.g., using amplicon sequencing) may also be obtained and used to determine a quality of the genetic variant. Advantages of using such a trained ML model include, but are not limited to, being a replacement for the QC process after the model is sufficiently trained and/or facilitating selection of genetic variants to include in the initial set of genetic variants for the panel based on the prediction of whether such variants would or would not pass the QC process.

To this end, some embodiments relate to using information from a QC process to train a machine learning model (e.g., a classification model) to estimate the quality of a genetic variant. FIG. 3B schematically illustrates a process 300 for training a machine learning model based on information obtained from a QC process in accordance with some embodiments. As shown in FIG. 3B, process 300 starts in act 310, where it is determined whether a particular genetic variant passed the QC process (e.g., whether the genetic variant was selectively detected in the tumour sample, indicating it is both somatic and amplified sufficiently for detection). If the genetic variant passed the QC process, the genetic variant may be classified as a good-quality variant in act 312. Otherwise, if the genetic variant did not pass the QC process, the genetic variant may be classified as a poor-quality variant in act 314. Having classified the genetic variant as good quality (act 312) or poor quality (act 314), process 300 proceeds to act 316, where a machine learning model may be trained using values for the genetic variant characteristics (e.g., determined in act 114 of process 100) and its classification (e.g., good-quality or poor-quality).

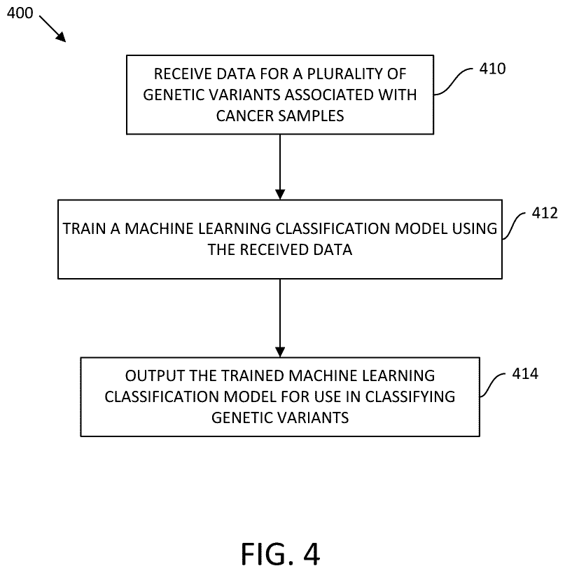

FIG. 4 illustrates a process 400 for training a machine learning classification model based on QC process data, in accordance with some embodiments of the present disclosure. Process 400 begins in act 410, where data for a plurality of genetic variants associated with cancer samples is received. For instance, the received data may include data obtained from a QC process, as described above in connection with FIGS. 3A and 3B. Table 1 describes an example of a dataset that may be received in act 410 obtained from 498 samples for different patients.

TABLE 1
Example dataset from QC process

| Variant Call | Good Quality | Poor Quality | Total |
|---|---|---|---|
| True Positive | 11546 | 1921 | 13467 |
| False Positive | 3754 | 2992 | 6746 |
| TOTAL | 15405 | 5149 | 20554 |

As shown in Table 1, the overall percentage of genetic variants that passed the QC process was approximately 66% indicating that the initial selection of genetic variants using existing (e.g., threshold-only based techniques) was incorrect about 34% of the time.

Process 400 then proceeds to act 412, where a machine learning (ML) classification model is trained using the received data. For instance, values for characteristics of the genetic variants included in the dataset shown in Table 1 may be provided as input to the ML classification model and a corresponding classification may be used as the corresponding output. The ML classification model may be trained to associate the values for characteristics with the corresponding classification. Process 400 then proceeds to act 414, where the trained ML classification model is output for use in classifying genetic variants.

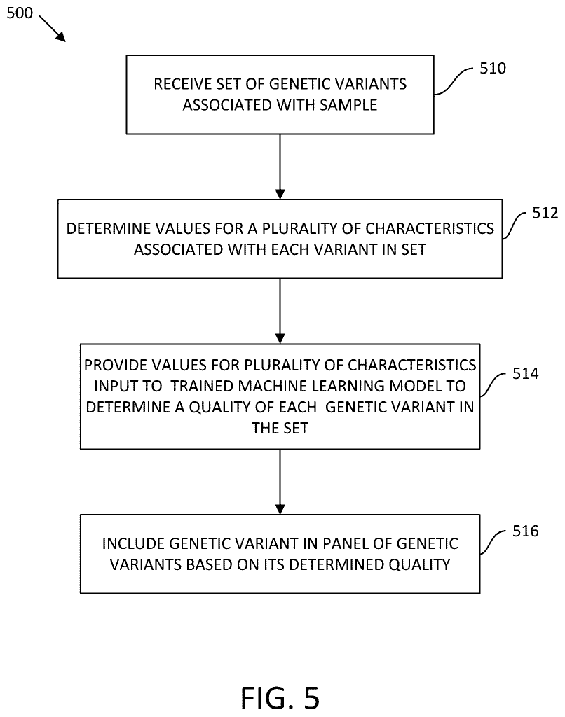

FIG. 5 illustrates a process 500 for using a trained ML model for determining a quality of a genetic variant, in accordance with some embodiments of the present disclosure. Process 500 begins in act 510, where a set of genetic variants associated with a sample is received. For instance, as described in connection with acts 110 and 112 of process 100, DNA sequencing (e.g., whole exome sequencing) may be performed on a tumour sample from a patient, and the resulting sequence reads may be aligned to the human reference genome. Genetic variants may be called to produce the set of genetic variants received in act 510. Process 500 then proceeds to act 512, where values for a plurality of characteristics associated with each genetic variant in the set may be determined. For example, as described in connection with act 114 of process 100, software tools such as Mutect2 and PureCN and/or other suitable software tools and/or algorithms may be used to identify somatic variants in the received set of variants, and values for a plurality of characteristics may be determined using such tools and/or algorithms. Process 512 then proceeds to act 514, where values for the plurality of characteristics for a particular genetic variant in the set are provided as input to a trained ML model, with the output of the trained ML model representing a quality of the genetic variant. In some embodiments, values for only a subset of the characteristics determined by the tools and/or algorithms may be provided as input to the trained ML model. The inventors have recognized that training a ML model on a smaller number of characteristics (e.g., 5-10 characteristics) may improve the accuracy of the model by, for example, preventing overfitting to the training data.

Process 500 then proceeds to act 516, where the genetic variant is included in a panel of genetic variants based on its determined quality. For instance, if the quality of the genetic variant is determined to be good, the genetic variant may be included in the panel, whereas if the quality of the genetic variant is determined to be poor, the genetic variant may not be included in the panel. In some embodiments, additional processing may occur prior to determining to include the genetic variant in the panel. For instance, the “good quality” genetic variants may be prioritized using one or more criteria, examples of which are described herein, and a subset of the good quality genetic variants (e.g., those having the highest priorities) may be included in the panel. In some embodiments, when a genetic variant is determined to have poor quality, it may not be included in the panel and may be replaced by another genetic variant. In other embodiments, poor quality variants simply may not be included in the panel, but not replaced provided that the panel includes a sufficient number of genetic variants remaining following exclusion of the poor quality variant from the panel. For example, if the initial set of genetic variants in consideration for panel inclusion includes 50 genetic variants and 10% of the variants are identified as poor quality genetic variants, those 5 variants may not be replaced and the panel may be proceed with just 45 genetic variants. The threshold number of variants to include in a panel may be configurable and set by a user. In some embodiments, when removal of the poor quality genetic variants would result in the total number of remaining genetic variants in the set being less than a threshold value, the entire panel may be considered poor quality and an indication of the poor quality of the panel may be output (e.g., to a user). The panel may then be redesigned prior to inclusion in the tumour-informed assay.

Any suitable ML classification model architecture may be trained using the techniques described herein. In some embodiments, the ML classification model is implemented using a random forest architecture. The inventors have recognized that use of random forest classification models may be particularly beneficial when it would be helpful to explain which characteristics or features contributed to a genetic variant being classified as a good quality genetic variant or a poor quality genetic variant. For instance, values for the predictors of a random forest classification model can be used to help a user understand why a genetic variant is likely to fail a QC process (e.g., be classified as a poor quality genetic variant). In some embodiments, information describing why a genetic variant is likely to fail a QC process may be output on a user interface and/or may be used to select an alternative variant that has different values for certain predictors in the model. In another example, the information output from the random forest classification model may provide insight into why a particular variant failed the QC process, and such information may be provided as feedback to a laboratory associated with the sample. In this way, information about the weighting of various predictors in the model to arrive at a classification for a particular genetic variant may be used to guide the selection process for genetic variants to use as replacements in the panel (e.g., selecting alternative variants). In some embodiments, the ML classification model may include one or more of a neural network (e.g., a deep learning neural network), or a gradient boosting decision trees model.

### Example Implementation of a Trained ML Model

In an example implementation of the techniques described herein, the dataset of genetic variants described in Table 1 was divided into a training set and a validation set (60/40 split) and a random forest classification model was trained on the training set to produce a trained ML classification model, as shown in Table 5. During a first training session, values for all available characteristics of the genetic variants were used to train the model. In a second training session, values for a subset of the available characteristics were used to train the random forest classification model. After both training sessions, the validation set of data was used to evaluate the accuracy of the trained ML model. Table 2 describes the results of predicting whether a genetic variant will fail or pass a QC process (e.g., is a poor quality or a good quality variant) after the random forest model was trained during the first training session, and Table 3 describes the predictive power for each characteristic used by the model.

TABLE 2
Random Forest Model Prediction Performance
(first training session)

| | Precision | Recall | Score | Number |
|---|---|---|---|---|
| Failed | 0.70 | 0.75 | 0.73 | 2698 |
| Passed | 0.87 | 0.84 | 0.85 | 5393 |
| Accuracy | | | 0.81 | 8091 |

TABLE 3
Model Characteristics (first training session)

| Characteristic | Predictive Power |
|---|---|
| ALT_T | 0.264230 |
| REF_T | 0.182571 |
| AF_T | 0.173137 |
| PureCN.ML.Expected_allelic_fraction | 0.114937 |
| posterior.somatic | 0.082720 |
| CN | 0.045315 |
| GERMQ | 0.044653 |
| insertion | 0.032009 |
| T_FILTER | 0.025252 |
| transition | 0.010735 |
| transversion | 0.010520 |
| is_snp | 0.006520 |
| PureCN.FLAGGED | 0.004508 |
| deletion | 0.002729 |
| EPR | 0.000164 |

As can be observed from Table 2, the prediction accuracy of 81% (reported as the number of correct predictions/number of predictions) using the trained random forest model is significantly better than the 66% accuracy of previous genetic variant selection techniques that rely solely on characteristic-based thresholds for variant selection (e.g., as shown in Table 1), thereby demonstrating an improvement in accuracy provided by using a trained ML model in accordance with some embodiments of the technology described herein.

As described above, during the second training session, a subset of the values for the available characteristics, which showed the most predictive value in the ML model trained during the first training session was used to assess whether training the model on fewer characteristics degraded the prediction accuracy of the model. Table 4 describes the results of predicting whether a genetic variant will fail or pass a QC process after the random forest model was trained during the second training session (here, using only eight characteristics which explained 95% of the predictive power, as shown in Table 5).

TABLE 4
Random Forest Model Prediction Performance
(second training session)

| | Precision | Recall | Score | Number |
|---|---|---|---|---|
| Failed | 0.70 | 0.74 | 0.72 | 2698 |
| Passed | 0.87 | 0.84 | 0.85 | 5393 |
| Accuracy | | | 0.81 | 8091 |

TABLE 5
Model Characteristics (second training session)

| Characteristic | Predictive Power |
|---|---|
| ALT_T | 0.284400 |
| REF_T | 0.193770 |
| AF_T | 0.185883 |
| PureCN.ML.Expected_allelic_fraction | 0.122140 |
| posterior.somatic | 0.086156 |
| CN | 0.045390 |
| GERMQ | 0.045161 |
| insertion | 0.037099 |

As can be observed from Tables 4 and 5, the prediction accuracy of 81% was the same as when the full set of characteristics was used to train the trained random forest model, indicating that training the ML model on a smaller set of characteristics may be sufficient to provide accurate prediction results. As described herein, some advantages of reducing the number of characteristics used to train the ML model may include, but are not limited to, reduced computational power (including more parameters may take a longer time to train) and a reduced risk of overfitting to the training data, which may reduce the generalization of the trained model. The risk of overfitting may be especially relevant for ML architectures more susceptible to overfitting such as neural networks or gradient boosting tree architectures.

In a further implementation example of the techniques described herein, an expanded dataset of genetic variants was divided into a training set and a validation set (60/40 split) and in a third training session a random forest classification model was trained on the training set to produce a trained ML classification model. This random forest classification model was additionally trained on characteristics including whether a given variant was likely to have been generated by exposure to one or more mutational signatures (e.g., mutational signatures associated with cancer). In the third training session, values for all available characteristics were used to train the model, and in a fourth training session, values for only a subset of the available characteristics were used to train the model. After both training sessions, the validation set of data was used to evaluate the accuracy of the trained ML model. Table 6 describes the results of predicting whether a genetic variant will fail or pass a QC process (e.g., is a poor quality or a good quality variant) after the random forest model was trained during the third training session, and Table 7 describes the predictive power of each characteristic used to train the model.

TABLE 6
Random Forest Model Prediction Performance
(third training session)

| | Precision | Recall | Score | Number |
|---|---|---|---|---|
| Failed | 0.74 | 0.78 | 0.76 | 5823 |
| Passed | 0.89 | 0.87 | 0.88 | 12136 |
| Accuracy | | | 0.84 | 17959 |

TABLE 7
Model Characteristics (third training session)

| Characteristic | Predictive Power |
|---|---|
| ALT_T | 0.249538 |
| REF_T | 0.181572 |
| AF_T | 0.164814 |
| prob(exposure to mutational signature) | 0.113188 |
| PureCN.ML.Expected_allelic_fraction | 0.091731 |
| posterior.somatic | 0.060873 |
| CN | 0.038242 |
| GERMQ | 0.037098 |
| T_FILTER | 0.030486 |
| Transition | 0.012453 |
| Transversion | 0.009337 |
| Insertion | 0.008456 |
| Deletion | 0.002213 |

As can be observed in Table 6, the prediction accuracy of 84% (reported as the number of correct predictions/number of predictions) is further improved over the 81% accuracy of the earlier examples that did not incorporate mutational signature characteristics (as shown in Tables 2-5), thereby demonstrating an improvement accuracy provided by using a trained ML model in accordance with some embodiments of the technology described herein.

During the fourth training session a subset of the values for the available characteristics, which showed the most predictive value in the ML model trained during the third training session, was used to assess whether training the model on fewer characteristics would be sufficient to provide accurate prediction results. In this training session, the characteristics comprising the top 80% of the total predictive value were selected, resulting in five characteristics to train the model. As shown in Table 8, reducing the number of characteristics resulted in a slight reduction in accuracy from 84% in the third training session to 83% in the fourth training session. However, as described herein, this reduction in accuracy may be offset by the capability of the model to generalize to new datasets, as opposed to overfitting to the training data set.

TABLE 8
Random Forest Model Prediction Performance
(fourth training session)

| | Precision | Recall | Score | Number |
|---|---|---|---|---|
| Failed | 0.74 | 0.76 | 0.75 | 5823 |
| Passed | 0.88 | 0.87 | 0.88 | 12136 |
| Accuracy | | | 0.83 | 17959 |

TABLE 9
Model Characteristics (fourth training session)

| Characteristic | Predictive Power |
|---|---|
| ALT_T | 0.440671 |
| REF_T | 0.227036 |
| AF_T | 0.189876 |
| prob(mutational signature) | 0.103044 |
| PureCN.ML.Expected_allelic_fraction | 0.039373 |

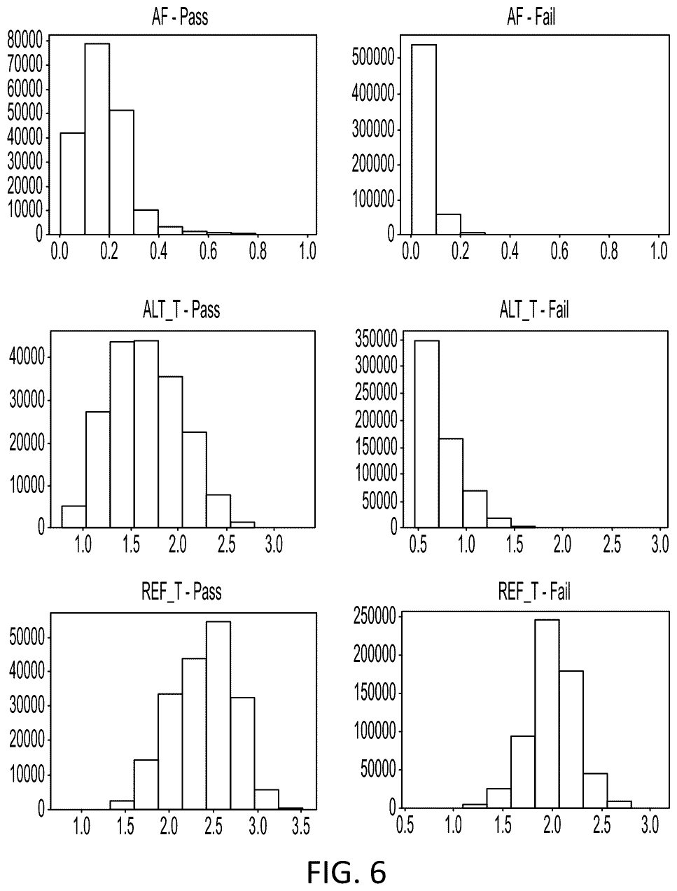

In another example, a random forest model according to the disclosure was generated and applied to a full variant dataset without any pre-filtering of variants. Most of the variants (76%) were predicted to be poor quality variants, in line with expectations based on the number of variants typically excluded by filtering and the number of variants that typically fail a QC process. FIG. 6 shows that this model produced sensible distributions between poor quality and good quality variants for characteristics such as allele frequency (AF), alternative allele depth (ALT_T), and reference allele depth (REF_T). As shown in FIG. 6, those variants predicted to be good quality variants (“Pass”) had generally higher values for these characteristics than those variants predicted to be poor quality variants (“Fail”). Accordingly, computational models as described herein can greatly simplify the variant selection process by omitting any additional variant filtering steps. The models can directly apply knowledge learned from previous datasets to entire unfiltered variant datasets and provide information about good quality and poor quality variants quickly and efficiently.

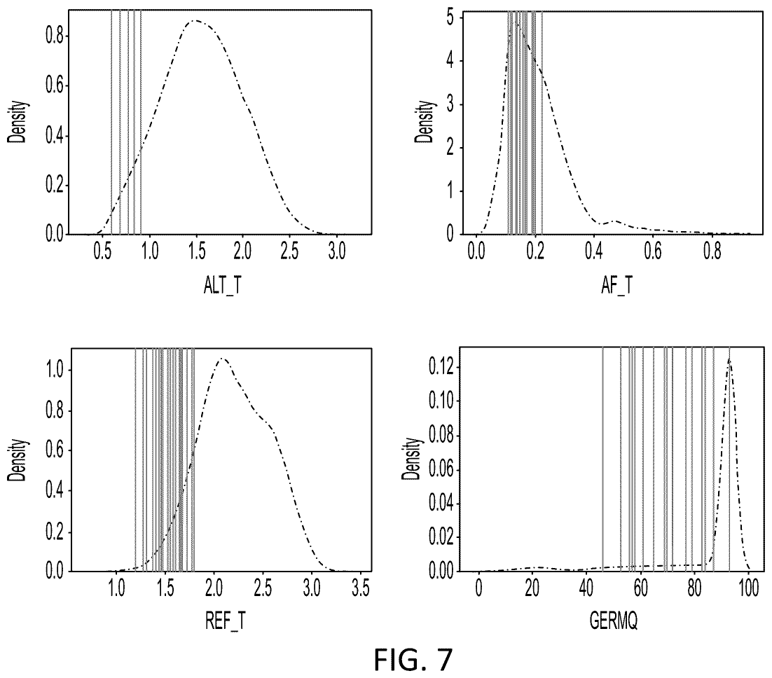

Distributions for the values of various characteristics for good quality and poor quality variants were then generated and compared. As shown in FIG. 7, a distribution was generated for all good quality variants for AF_T, ALT_T, REF_T, and GERMQ and visualized in a density plot. Poor quality variants are represented as vertical lines. Generally, the poor quality variants cluster at lower values than the mean for the good quality variants for each characteristic. For example, as shown in FIG. 7, predicted poor quality variants have ALT_T, GERMQ, and REF_T values much lower than what would be expected for a good quality variant. For AF_T, the poor quality variants are close to the average for the good quality variants; however, there are a significant number of good quality variants having values higher than the poor quality variants.

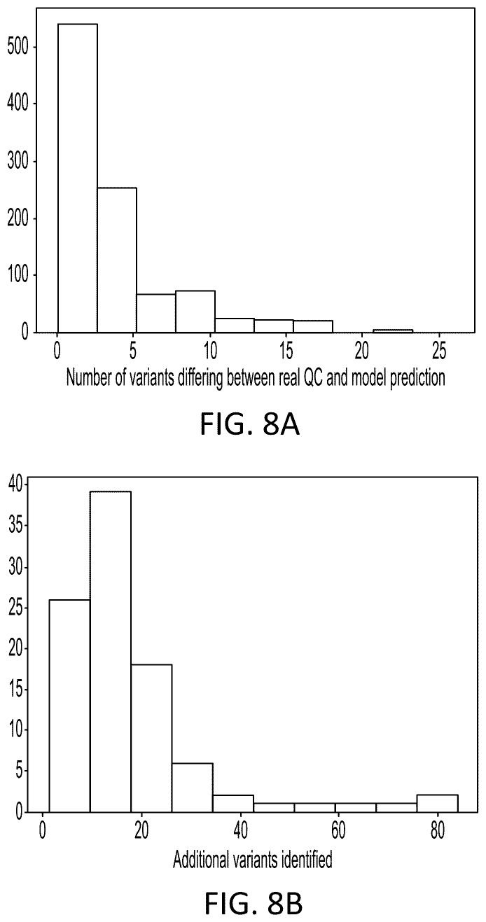

Computational models as described herein may be used to partially or entirely replace a QC process. In another example, 48-variant panels with data from a QC process were compared with corresponding predictions from a trained model (FIG. 8A). The results were highly concordant. Over 75% of the panels had a different result for only five or less variants, showing that the model predictions are highly similar to a QC process. Next, the trained model was used to identify additional variants for inclusion in previously-generated panels which had failed the QC process, e.g., less than eight variants were identified as good quality variants (FIG. 8B). The model identified an average of 13 additional variants for each panel (from other variants not included on the panel), thereby rescuing the panel and rendering it suitable for use with a patient sample.

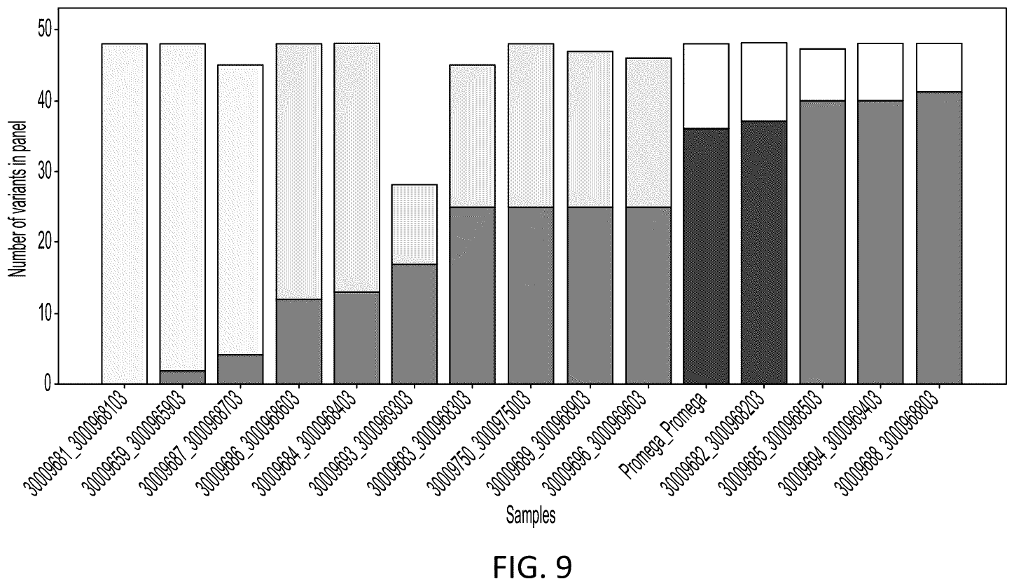

FIG. 9 illustrates another example of how computational models described herein can guide panel design and be substituted, at least in part, for a QC process when selecting genetic variants for inclusion in a panel. In this example, 15 patient panels, each having between 25-48 variants, were randomly selected and a computational model was applied to predict how many variants were poor quality (light tone) vs. good quality (dark tone) in each sample. As shown in FIG. 9, from left to right, three of the panels were predicted to have less than nine good quality variants; three of the panels were predicted to have 10-19 good quality variants; four of the panels were predicted to have 20-29 good quality variants; two of the panels were predicted to have 30-39 good quality variants; and three of the panels were predicted to have at least 40 good quality variants. This information could be used to inform a decision of whether to use a panel on a patient sample or replace any genetic variants on the panel with alternative genetic variants not initially included on the panel to render it suitable for using with a patient sample, without performing an additional QC process. Accordingly, models according to the disclosure can greatly simplify variant selection for panels by directly applying the knowledge learned from previous datasets and QC processes to select good quality variants for inclusion.

Having thus described several aspects and embodiments of the technology set forth in the disclosure, it is to be appreciated that various alterations, modifications, and improvements will readily occur to those skilled in the art.

Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more hard drives, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of assessing quality of a genetic variant for inclusion as a biomarker in a circulating tumour DNA (ctDNA) assay, the method comprising:
receiving a set of genetic variants associated with a sample collected from a patient, the set of genetic variants including a first genetic variant;
determining values for a plurality of characteristics associated with the first genetic variant;
providing the values for the plurality of characteristics as input to a trained machine learning (ML) model, the trained ML model being trained to output a quality of a genetic variant, the quality of the genetic variant representing a likelihood that the genetic variant is both somatic and will sufficiently amplify using amplicon sequencing; and
including the first genetic variant in a panel of genetic variants for use in a ctDNA assay for the patient based on the quality of the first genetic variant output from the trained ML model.

2. The method of claim 1, wherein the trained ML model is a classification model trained to classify the genetic variant as a good genetic variant or a poor genetic variant, wherein classification as a good genetic variant represents a high likelihood that the genetic variant is both somatic and will sufficiently amplify using amplicon sequencing.

3. The method of claim 2, wherein including the first genetic variant in a panel of genetic variants for use in a ctDNA assay for the patient based on the quality of the first genetic variant output from the trained ML model comprises including the first genetic variant when the first genetic variant is classified as a good genetic variant.

4. The method of claim 2, wherein the trained ML model includes one or more of a random forest model, a neural network, or a gradient boosting decision trees model.

5. The method of claim 2, wherein including the first genetic variant in a panel of genetic variants for use in a ctDNA assay for the patient based on the quality of the first genetic variant output from the trained ML model comprises substituting a second genetic variant for the first genetic variant in the panel of genetic variants when the first genetic variant is classified as a poor genetic variant.

6. The method of claim 5, further comprising:
determining values for the plurality of characteristics for the second genetic variant;
providing, for the second genetic variant, corresponding values for the plurality of characteristics as input to the trained ML model to determine a quality of the second genetic variant; and
substituting the second genetic variant for the first genetic variant in the panel of genetic variants only when the quality of second genetic variant is classified as a good genetic variant.

7. The method of claim 2, wherein when the first genetic variant is classified as a poor genetic variant, the method further comprises:
outputting an indication of one or more reasons why the first genetic variant was classified as a poor genetic variant.

8. The method of claim 2, further comprising:
determining values for the plurality of characteristics for each of the genetic variants in the set of genetic variants;
providing, for each of the genetic variants in the set of genetic variants, corresponding values for the plurality of characteristics as input to the trained ML model to determine a quality of the genetic variant; and
outputting an indication that the set of genetic variants is of poor quality when more than a threshold number of genetic variants in the set is determined to have a poor quality.

9. The method of claim 1, further comprising:
determining a number of genetic variants in the panel of genetic variants; and
removing the first genetic variant from the panel of genetic variants when the quality of the genetic variant is determined to have a poor quality and the number of genetic variants in the panel is greater than a threshold number.

10. The method of claim 1, wherein the plurality of characteristics include one or more of read depth characteristics, copy number characteristics, variant quality characteristics, posterior somatic probability characteristics, or mutational signature characteristics.

11. The method of claim 1, further comprising:
filtering the set of genetic variants to produce a filtered set of genetic variants, wherein the first genetic variant is included in the filtered set of genetic variants.

12. The method of claim 1, wherein the sample is a cancer sample.

13. The method of claim 1, wherein the ctDNA assay is an amplicon sequencing assay.

14. The method of claim 1, further comprising:
testing a plasma sample from the patient using the ctDNA assay; and
outputting an assay result.

15. The method of claim 14, wherein the plasma sample is a sample taken 1-12 months after surgery to remove cancerous tissue from the patient.

16. A system, comprising:
at least one hardware computer processor programmed to:
receive a set of genetic variants associated with a sample collected from a patient, the set of genetic variants including a first genetic variant;
determine values for a plurality of characteristics associated with the first genetic variant;
provide the values for the plurality of characteristics as input to a trained machine learning (ML) model, the trained ML model being trained to output a quality of a genetic variant, the quality of the genetic variant representing a likelihood that the genetic variant is both somatic and will sufficiently amplify using amplicon sequencing; and
include the first genetic variant in a panel of genetic variants for use in a ctDNA assay for the patient based on the quality of the first genetic variant output from the trained ML model.

17. The system of claim 16, wherein the trained ML model is a classification model trained to classify the genetic variant as a good genetic variant or a poor genetic variant, wherein classification as a good genetic variant represents a high likelihood that the genetic variant is both somatic and will sufficiently amplify using amplicon sequencing.

18. The system of claim 17, wherein the at least one hardware computer processor is further programmed to:
determine values for the plurality of characteristics for each of the genetic variants in the set of genetic variants;
provide, for each of the genetic variants in the set of genetic variants, corresponding values for the plurality of characteristics as input to the trained ML model to determine a quality of the genetic variant; and
output an indication that the set of genetic variants is of poor quality when more than a threshold number of genetic variants in the set is determined to have a poor quality.

19. The system of claim 16, wherein when the first genetic variant is classified as a poor genetic variant, the at least one hardware computer processor is further programmed to:
output an indication of one or more reasons why the first genetic variant was classified as a poor genetic variant.

20. A computer readable medium encoded with a plurality of instructions, that, when executed by at least one hardware computer processor perform a method of:
receiving a set of genetic variants associated with a sample collected from a patient, the set of genetic variants including a first genetic variant;
determining values for a plurality of characteristics associated with the first genetic variant;
providing the values for the plurality of characteristics as input to a trained machine learning (ML) model, the trained ML model being trained to output a quality of a genetic variant, the quality of the genetic variant representing a likelihood that the genetic variant is both somatic and will sufficiently amplify using amplicon sequencing; and
including the first genetic variant in a panel of genetic variants for use in a ctDNA assay for the patient based on the quality of the first genetic variant output from the trained ML model.
